(19) United States
(12) Patent Application Publication
(10) Pub. No.: US 2025/0235189 A1
(43) Pub. Date: Jul. 24, 2025

(54) BIOPSY DEVICE

(71) Applicant: Eddy Pump Corporation, El Cajon, CA (US)

(72) Inventors: Daniel WAHLGREN, Escondido, CA (US); Roberto GONZALEZ, Chula Vista, CA (US); Hugo Miranda CORDOVA, Jalisco (MX); Sam BARADARIAN, San Diego, CA (US)

(21) Appl. No.: 19/034,751

(22) Filed: Jan. 23, 2025

Related U.S. Application Data
(60) Provisional application No. 63/624,435, filed on Jan. 24, 2024.

(51) Int. Cl. A61B 10/02

(52) U.S. Cl. CPC A61B 10/0266

(57) ABSTRACT

Biopsy devices and methods are disclosed herein. In an embodiment, a biopsy device includes a hollow shaft, a cutting member and an engagement member. The cutting member is connected to an end of the hollow shaft and configured to cut into tissue. The engagement member is movably connected to the hollow shaft and configured to move rotationally and axially relative to the cutting member. The engagement member is configured to move from a first position to a second position engaging the cutting member after the cutting member has initially engaged the tissue.

### BACKGROUND

### Priority

This application claims priority to U.S. Provisional Application No. 63/624,435, filed Jan. 24, 2024 and entitled “Biopsy Device,” the entire contents of which is incorporated herein by reference.

### FIELD

The present disclosure generally relates to biopsy devices and corresponding methods of taking tissue samples.

### BACKGROUND

Biopsy procedures, such as lung biopsy procedures, are performed thousands of times per day worldwide. Currently, the most common form of lung biopsy involves hospitalization for two to three days, thoracic surgical expertise, general anesthesia, usually requiring double lumen intubation for lung isolation, video-assisted thoracoscopic surgery (VATS) or thoracotomy approach, often requiring several days of recovery, with postoperative complications including but not limited to air leak, post procedure pain control requiring narcotics, and prolonged hospitalization.

Interstitial lung disease (ILD) is one of many diseases affecting lung parenchyma, specifically the interstitium, which is the connective tissue between alveoli. It is described as a progressively fibrotic process, which ultimately leads to lung failure, and a significant contributor of morbidity and mortality in the United States and worldwide. In 2019, in the United States, there were an estimated 654,500 cases of ILD diagnosed, costing almost 450,000 disability adjusted life years, and 21,500 deaths. Computed tomography (CT) scan is emerging as a reliable diagnostic tool, but histopathologic evaluation by biopsy remains the standard for specificity. Traditional minimally invasive biopsy tools, such as fine needle aspiration, or core needle biopsy, do not yield large enough specimens for detailed parenchymal or interstitial architectural analysis, as a specimen is required to be at least between five mm to ten mm in diameter.

### SUMMARY

The present disclosure provides devices and methods that reduce the difficulty, complexity, cost, and invasiveness of biopsy procedures and increase the efficiency of obtaining a tissue sample for histopathologic evaluation.

In view of the state of the known technology, a first aspect of the present disclosure is to provide a biopsy device including a hollow shaft, a cutting member and an engagement member. The cutting member is connected to an end of the hollow shaft and configured to cut into tissue. The engagement member is movably connected to the hollow shaft and configured to move rotationally and axially relative to the cutting member. The engagement member is configured to move from a first position to a second position engaging the cutting member after the cutting member has initially engaged the tissue.

A second aspect of the present disclosure is to provide a method of performing a biopsy. The method includes cutting into tissue with a cutting member attached to a hollow shaft at a location of a desired tissue sample, moving an engagement member into the engagement with the cutting tool so as to further sever the tissue sample from the tissue, and withdrawing the tissue sample through the hollow shaft with a tissue removal tool after the tissue sample is severed from the tissue by interaction of the cutting member and the engagement member.

A third aspect of the present disclosure is to provide a biopsy device including a first gripping member, a second gripping member, a cutting tool and at least one staple. The second gripping member is separated from the first gripping member by an axially extending gap. The cutting tool is adjacent one of the first gripping member and the second gripping member. The at least one staple is disposed in one of the first and second gripping members. The first gripping member and the second gripping member are positioned and arranged such that movement of at least one of the first gripping member and the second gripping member toward the other with tissue located in the axially extending gap both removes a tissue sample from the tissue and ejects the staple into the tissue.

Also, other objects, features, aspects and advantages of the disclosed biopsy devices and methods will become apparent to those skilled in the art from the following detailed description, which, taken in conjunction with the annexed drawings, discloses several embodiments of biopsy devices and methods.

### BRIEF DESCRIPTION OF THE DRAWINGS

Referring now to the attached drawings which form a part of this original disclosure:

FIG. 1 is a perspective view of an example embodiment of a biopsy device in accordance with the present disclosure;

FIG. 2 is a perspective view of the cutting tool of the biopsy device of FIG. 1;

FIG. 3 is another perspective view of parts of the cutting tool of the biopsy device of FIG. 1;

FIG. 4 is another perspective view of parts of the cutting tool of the biopsy device of FIG. 1;

FIG. 5 is another perspective view of parts of the cutting tool of the biopsy device of FIG. 1;

FIG. 6 is a perspective view of the cutting member of the biopsy device of FIG. 1;

FIG. 7 is a perspective view of the engagement member of the biopsy device of FIG. 1;

FIG. 8 is a perspective view of the cutting mechanism formed by the cutting member and the engagement member of the biopsy device of FIG. 1;

FIG. 9 is a perspective view of the sample removal tool of the biopsy device of FIG. 1;

FIG. 10 is a perspective view of an example embodiment of another sample removal tool;

FIG. 11 is a perspective view of an example embodiment of another sample removal tool;

FIG. 12 is a perspective view of an example embodiment of another sample removal tool;

FIG. 13 is a perspective view of an example embodiment of another sample removal tool;

FIG. 14 is a perspective view of an example embodiment of another sample removal tool;

FIG. 15 is a schematic illustration of the biopsy device of FIG. 1 being used to remove a tissue sample;

FIG. 16 is a schematic illustration of the biopsy device of FIG. 1 being used to remove a tissue sample;

FIG. 17 is a schematic illustration of the biopsy device of FIG. 1 being used to remove a tissue sample;

FIG. 18 is a schematic illustration of the biopsy device of FIG. 1 being used to remove a tissue sample;

FIG. 19 is a schematic illustration of the biopsy device of FIG. 1 being used to remove a tissue sample;

FIG. 20 is a schematic illustration of the biopsy device of FIG. 1 being used to remove a tissue sample;

FIG. 21 is a detailed perspective view of the cutting mechanism of the biopsy device of FIG. 1;

FIG. 22 is another detailed perspective view of the cutting mechanism of the biopsy device of FIG. 1;

FIG. 23 is another detailed perspective view of the cutting mechanism of the biopsy device of FIG. 1;

FIG. 24 is a cross-sectional view of the cutting member of the biopsy device of FIG. 1;

FIG. 25 is another cross-sectional view of the cutting member of the biopsy device of FIG. 1;

FIG. 26 is a cross-sectional view of the engagement member of the biopsy device of FIG. 1;

FIG. 27 is another cross-sectional view of the engagement member of the biopsy device of FIG. 1;

FIG. 28 is a perspective view of a biopsy system including the biopsy device of FIG. 1;

FIG. 29 is a perspective view of another embodiment of the cutting mechanism of a biopsy device in accordance with the present disclosure;

FIG. 30 is a side elevational view of the cutting mechanism of FIG. 28;

FIG. 31 is another side elevational view of the cutting mechanism of FIG. 28;

FIG. 32 is a perspective view of the cutting mechanism of FIG. 28;

FIG. 33 is another perspective view of the cutting mechanism of FIG. 28;

FIG. 34 is a perspective view of another embodiment of a biopsy device in accordance with the present disclosure;

FIG. 35 is a side elevational view of the biopsy device of FIG. 34;

FIG. 36 is a partial perspective view of the biopsy device of FIG. 34;

FIG. 37 is a perspective view of another embodiment of a biopsy device in accordance with the present disclosure;

FIG. 38 is a side elevational view of a staple used by the biopsy devices in accordance with the present disclosure;

FIG. 39 is a side elevational view of the staple of FIG. 38 in a closed configuration; and

FIG. 40 is a side elevational view of the staple of FIG. 38 closing tissue.

Throughout the drawing figures, like reference numerals will be understood to refer to like parts, components and structures.

### DETAILED DESCRIPTION

Selected exemplary embodiments will now be explained with reference to the drawings. It will be apparent to those skilled in the art from this disclosure that the following descriptions of the exemplary embodiments are provided for illustration only and not for the purpose of limiting the invention as defined by the appended claims and their equivalents.

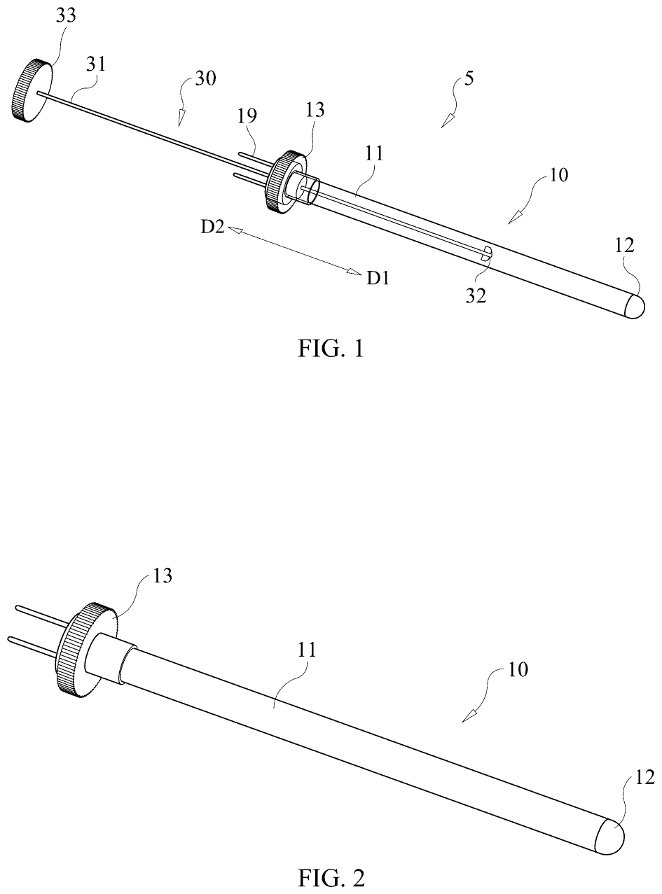

FIG. 1 illustrates a first example embodiment of a biopsy device 5 in accordance with the present disclosure. In the illustrated embodiment, the biopsy device 5 includes a cutting tool 10 and a sample removal tool 30. As discussed in more detail below, the sample removal tool 30 translates within the cutting tool 10 in the directions D1 and D2 to remove a tissue sample TS from tissue T (e.g., from lung tissue) during a biopsy.

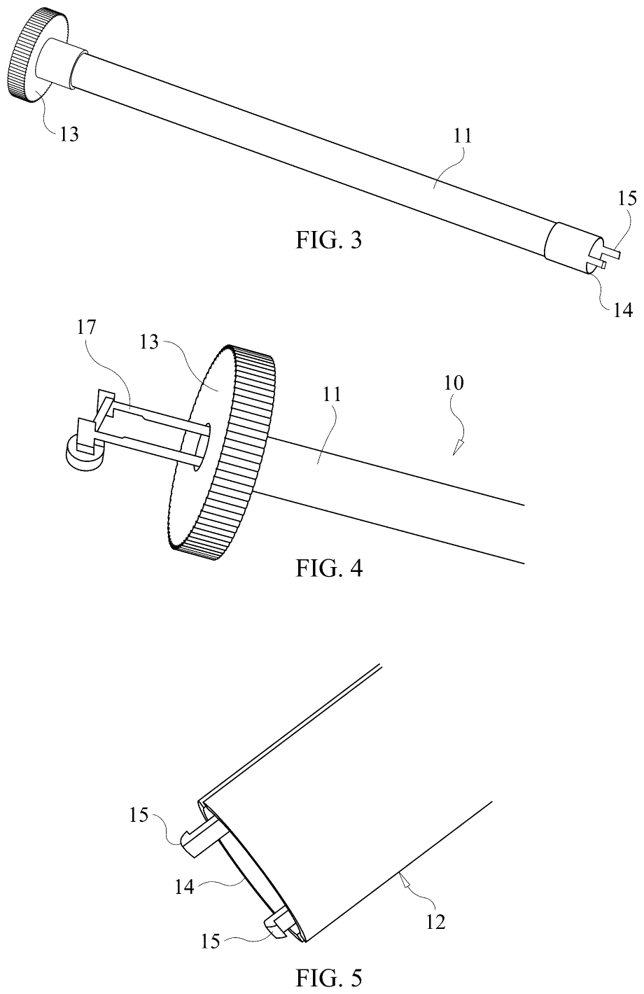

FIGS. 2 to 8 illustrate an example embodiment of the cutting tool 10 in more detail. In the illustrated embodiment, the cutting tool 10 includes a hollow shaft 11 (also referred to as an “introducer port”), a cutting mechanism 12 and a handling mechanism 13. The cutting mechanism 12 and the handling mechanism 13 are located at opposite ends of the hollow shaft 11. The tissue removal tool 30 translates within the hollow shaft 11 in the direction D1 to be placed in position within the cutting mechanism 12 to retrieve a tissue sample TS, and in the direction D2 to remove the tissue sample TS from the hollow shaft 11 after the tissue sample TS is severed from the tissue T by the cutting mechanism 12.

The cutting mechanism 12 cuts the tissue sample TS from the tissue T so that the tissue removal tool 30 can withdraw the tissue sample TS in the direction D2 for further analysis. In the illustrated embodiment, the cutting mechanism 12 includes a cutting member 16 and an engagement member 18. As discussed in more detail below, the cutting member 16 is located at the end of the hollow shaft 11 and makes the initial cut into the tissue T when the cutting tool 10 is used during a biopsy. The engagement member 18 translates within the hollow shaft 11 and rotates into engagement with the cutting member 16. After the cutting member 16 has made the initial cut into the tissue T, the engagement member 18 rotates into the cutting member 16 to further sever and release a tissue sample TS for removal by the tissue removal tool 30 and/or to further cause the cutting mechanism 12 to anchor in the tissue T.

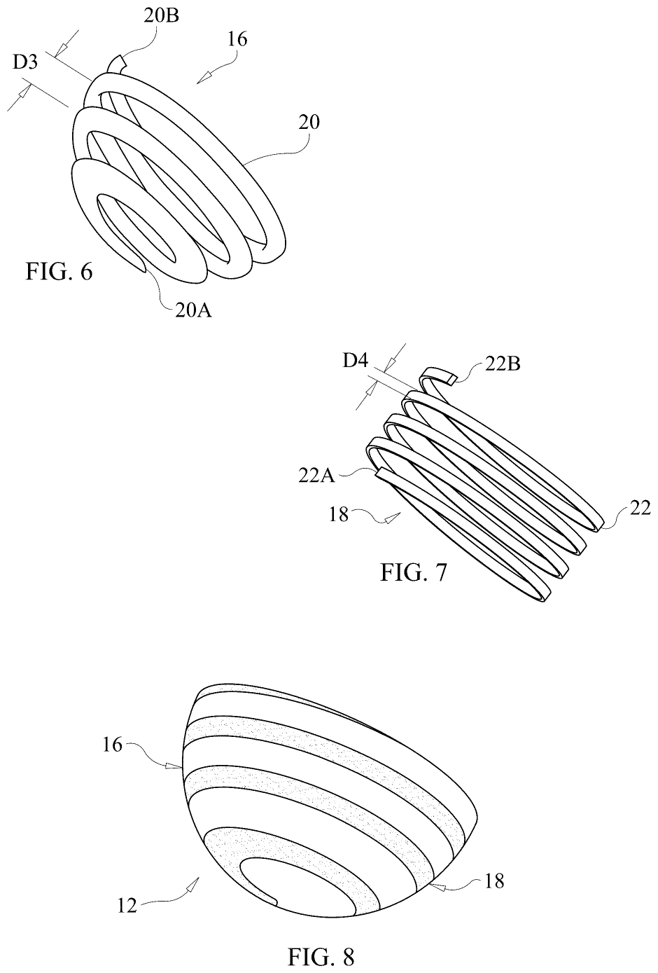

FIG. 6 illustrates an example embodiment of the cutting member 16. In the illustrated embodiment, the cutting member 16 is an integrally formed single strand 20 of metal, for example, a non-ferromagnetic material such as titanium. The strand 20 preferably has a substantially helical shape extending from a first end 20A to a second end 20B. Edges of the strand 20 are sharp to facilitate moving the cutting member 16 through tissue T. As seen in FIG. 6, a space of distance D3 is defined between adjacent positions on successive coils of the strand 20. In an embodiment, each successive coil of the strand 20 has a larger outer diameter to form the substantially helical shape.

FIG. 7 illustrates an example embodiment of the engagement member 18. In the illustrated embodiment, the engagement member 18 is also an integrally formed single strand 22 of metal, for example, a non-ferromagnetic material such as titanium. The strand 22 preferably has a substantially cylindrical shape extending from a first end 22A to a second end 22B. As seen in FIG. 7, each successive coil of the strand 22 has a substantially equivalent outer diameter to form a substantially cylindrical shape. The strand 22 shown in FIG. 7 has a thickness of distance D4, which generally corresponds to the space of distance D3 of the cutting member 16, enabling the engagement member 18 to rotate into the cutting member 16, for example, as seen in FIGS. 8 and 17 to 20. In the illustrated embodiment, as the engagement member 18 rotates into the cutting member 16, the overall shape of the engagement member 18 conforms to the shape of the cutting member 16 to form the cutting mechanism 12 in the shape shown in FIG. 8.

The handling portion 13 enables the user to handle and/or control rotation of one or more elements of the cutting tool 10. The handling portion 13 can be held by a user when a biopsy is taken using the cutting tool 10. In an embodiment, the handling portion 13 rotates with respect to the hollow shaft 11 or with respect to another element within the hollow shaft 11 to cause the engagement member 18 to rotate with respect to the cutting member 16. In another embodiment, the handling portion 13 and the hollow shaft 11 rotate together.

As seen in FIGS. 2 to 5, the hollow shaft 11 includes a distal end 14 opposite the handling portion 13. The distal end includes two arms 15 which protrude from the distal end for attachment of the cutting mechanism 12. As seen in FIG. 4, the arms 15 can be attached to one or more projection 17 which extends through the hollow shaft 11. In an embodiment, one of the cutting member 16 and the engagement member 18 is attached to the arms 15 at the distal end 14. For example, the cutting member 16 can be attached to the hollow shaft 11 at the distal end 14, and the engagement member 18 can be connected to the arms 15, or vice versa. A user can then move the engagement member 18 with respect to the cutting member 16 be translating and/or rotating the projection 17 or projections 19 (FIG. 1) with respect to the hollow shaft 11.

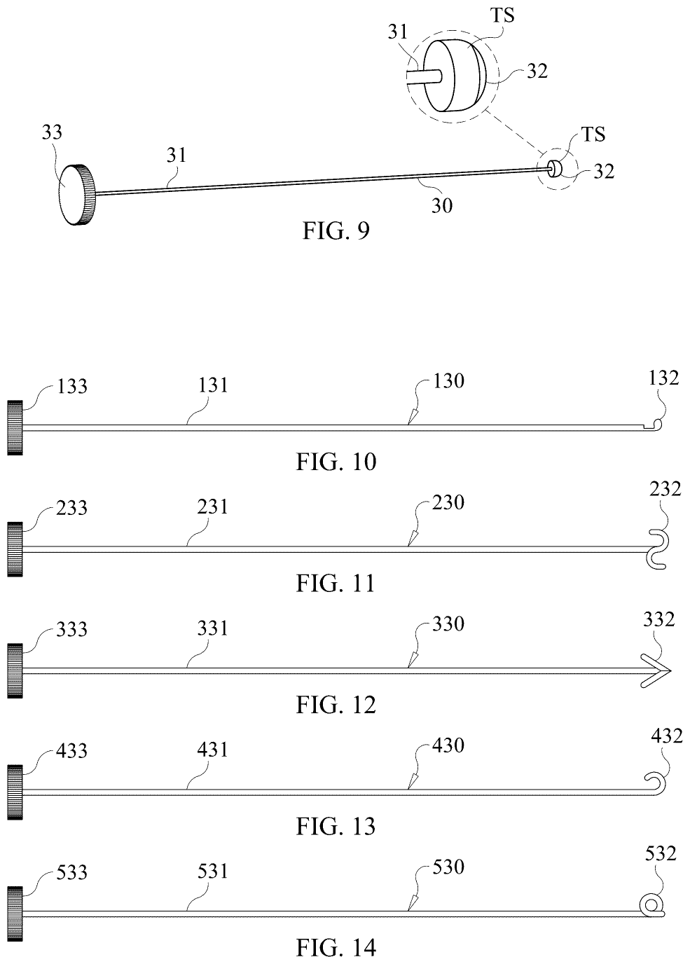

FIG. 9 illustrates a first example embodiment of the tissue removal tool 30. In the illustrated embodiment, the tissue removal tool 30 includes an elongated shaft 31, a tissue sample retainer 32, and a handling portion 33. In an embodiment, the tissue sample retainer 32 is a substantially arcuate member. The tissue sample retainer 32 moves axially through the hollow shaft 11 in the direction D1 to engage a tissue sample TS removed from the tissue T by the cutting mechanism 12. For example, the tissue sample retainer 32 can include one or more hook or hook-like shape that engages and securely retains the tissue sample TS for withdrawal in the direction D2 and removal from the hollow shaft 11 for analysis.

FIGS. 10 to 14 illustrate several alternative embodiments of the tissue removal tool 30. In FIG. 10, the tissue removal tool 130 includes an elongated shaft 131, a tissue sample retainer 132, and a handling portion 133, with the tissue sample retainer 132 including a substantially pig-tail shaped hook. In FIG. 11, the tissue removal tool 230 includes an elongated shaft 231, a tissue sample retainer 232, and a handling portion 233, with the tissue sample retainer 232 including a substantially S-shaped hook. In FIG. 12, the tissue removal tool 330 includes an elongated shaft 331, a tissue sample retainer 332, and a handling portion 333, with the tissue sample retainer 332 including an arrow-shaped hook. In FIG. 13, the tissue removal tool 430 includes an elongated shaft 431, a tissue sample retainer 432, and a handling portion 433, with the tissue sample retainer 432 including a substantially 180-degree coil hook. In FIG. 14, the tissue removal tool 530 includes an elongated shaft 531, a tissue sample retainer 532, and a handling portion 533, with the tissue sample retainer 532 including a substantially 360-degree coil hook. Those of ordinary skill in the art will recognize from this disclosure that the tissue sample retainer 32 can also be formed of other shapes that are capable of withdrawing a tissue sample TS as discussed herein.

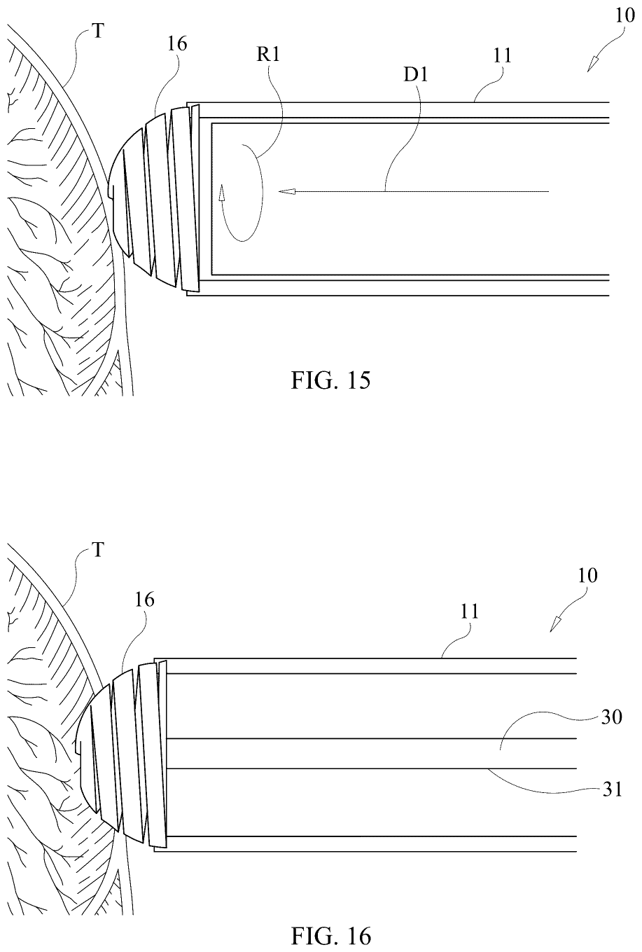

FIGS. 15 to 20 illustrate an example embodiment of the biopsy device 5 being used to remove a tissue sample TS from tissue T (e.g., lung tissue). Briefly summarized, the cutting member 16 is advanced around the tissue removal tool 30 by rotating the cutting tool 10. The engagement member 18 can include sharpened edges and be axially and rotatably moved through the hollow shaft 11 to engage the cutting member 16. As the engagement member 18 is guided along the cutting member 16, the tissue sample TS is severed and released from the tissue T. The engagement member 18 can move into engagement with the cutting member 16 until being stopped from further axial movement by the cutting member 16 to form the completed cutting mechanism 12 as shown in FIG. 8. The tissue removal tool 30 can then withdraw the severed tissue sample TS from the cutting tool 10 for further analysis.

The method also generally includes identification of a target area of tissue T and local analgesia obtained on chest wall skin, through subcutaneous tissues, and above a desired rib, to avoid intercostal artery, vein and nerve. A Seldinger technique can be used to gain access into pleural space, and the chest wall tract can be dilated over the tissue sample retainer 32. The tissue removal tool 30 is introduced into the tissue T through the cutting tool 10 to secure and withdraw the tissue sample TS.

FIG. 15 illustrates the cutting tool 10 initially engaging the tissue T. Here, the cutting tool 10 is moved in the direction D1 into contact with the tissue T to be sampled. The cutting member 16 is then rotated in the rotational direction R1 to cause the sharpened edges of the cutting member 16 to cut into the tissue T. In an embodiment, the cutting member 16 is rotated by rotating the hollow shaft 11 and/or handling portion 13. In another embodiment, the cutting member 16 is rotated by rotating the protrusion 17, protrusions 19 and/or another element.

FIG. 16 illustrates the cutting tool 10 after the cutting member 16 has made an initial cut into the tissue T. In FIG. 16, the tissue removal tool 30 is advanced through the hollow shaft 11 so that the tissue sample retainer 32 is located within the inner space of the cutting member 16. In another embodiment, the tissue sample retainer 32 can be located within the inner space of the cutting member 16 while the cutting member 16 makes the initial cut illustrated in FIG. 15.

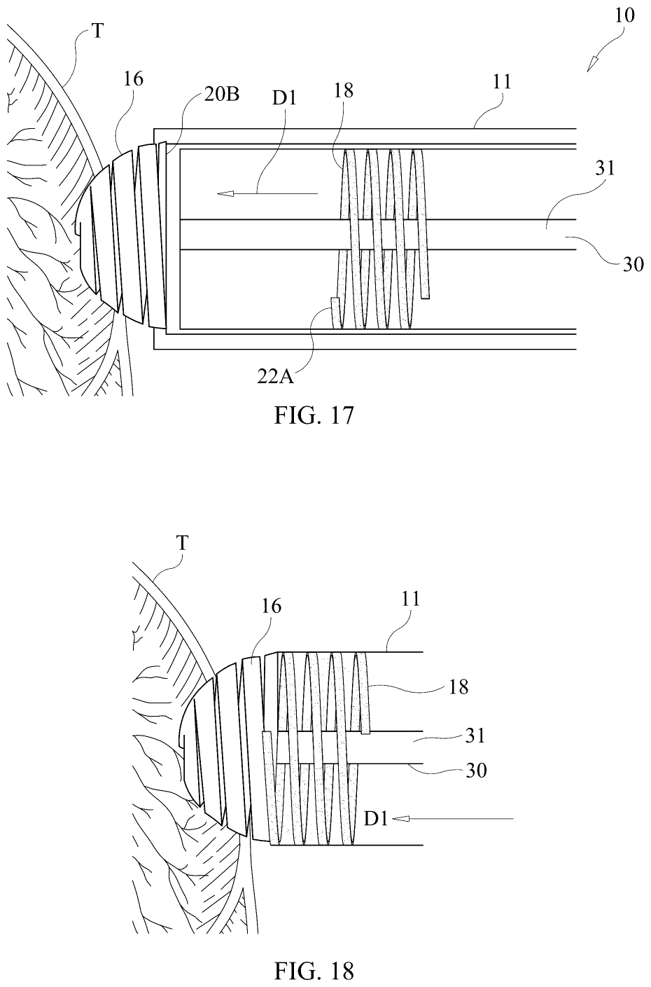

FIG. 17 illustrates the engagement member 18 being advanced axially in the direction D1 towards the cutting member 16. Here, the engagement member 18 encircles the elongated shaft 31 of the tissue removal tool 30, which enables the engagement member 18 to advance within the hollow shaft 11 independently of the tissue removal tool 30. In an embodiment, the engagement member 18 is advanced by moving the protrusion 17 or protrusions 19 with respect to the hollow shaft 11. The engagement member 18 does not need to advance the entire length of the hollow shaft 11 and can also initially be located adjacent the cutting member 16.

FIG. 18 illustrates the engagement member 18 beginning to engage the cutting member 16 by rotating into the cutting member 16. In an embodiment, the cutting member 16 and the engagement member 18 can be positioned in this or a similar configuration when the cutting member 16 initially cuts into the tissue T instead of the engagement member 18 translating through the hollow shaft as shown in FIG. 17.

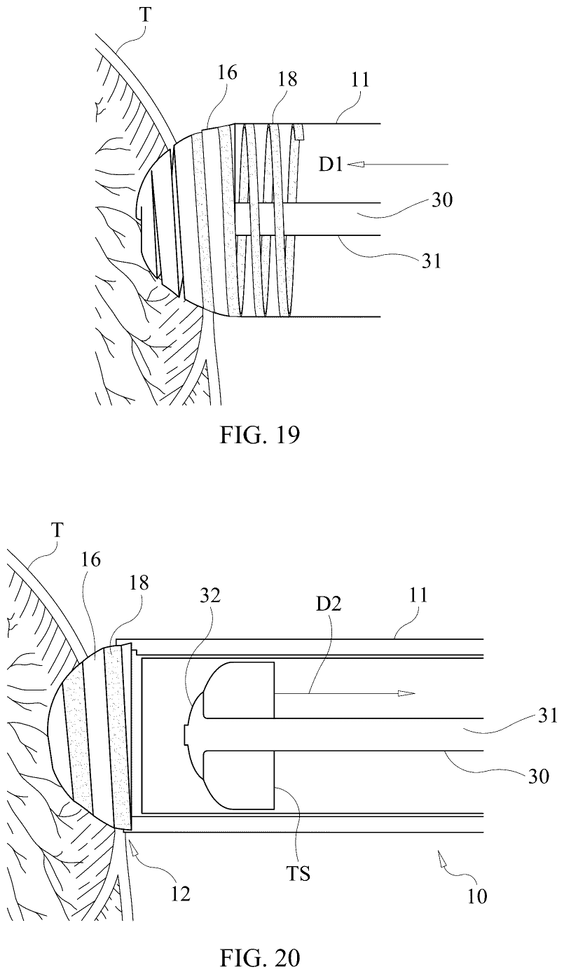

FIG. 19 illustrates the engagement member 18 having rotated further into the cutting member 16. As the engagement member 18 rotates, the strand 22 of the engagement member 18 fills in the gaps in the cutting member 16 (e.g., the space of distance D3 shown in FIG. 6) and severs the remaining tissue T corresponding to those gaps that was not cut by the cutting member 16. In other words, the cutting member 16 partially cuts the tissue T and anchors the cutting tool 10 in the tissue T, and the engagement member 18 finishes the cutting and/or anchoring, severing the remaining part of the tissue T the surrounding cutting mechanism 12. In the illustrated embodiment, the engagement member 18 rotates into the cutting member 16 until the engagement member 18 can rotate no more, for example, when the cutting member 16 and the engagement member 18 form the configuration of the cutting mechanism 12 shown in FIG. 8.

In the illustrated embodiment, the engagement member 18 initially moves into engagement with the cutting member 16 when the first end 22A of the engagement member 18 engages the second end 20B of the cutting member 16. The engagement member 18 then continues to move axially and rotationally, such that the engagement member 18 is guided by the cutting member 16. The engagement member 18 continues to move axially and rotationally until the first end 22A of the engagement member 18 contacts a stop on the cutting member 16, thereby stopping further movement of the engagement member 18 in the distal direction (e.g., the direction D1). In other words, further movement of the engagement member 18 toward the tissue T is stopped. When the engagement member 18 reaches the stop, the cutting mechanism 12 is fully formed. The thickness of the strand 22 of the engagement member 18 fills in the gaps between the successive coils of the strand 20 of the cutting member 16. As shown in FIGS. 7, 17 and 18, in a first position spaced from the cutting member 16, the engagement member 18 has a substantially cylindrical shape. As shown in FIGS. 8 and 20, in a second position engaged with the cutting member 16, the engagement member 18 has a substantially helical shape corresponding to the shape of the cutting member 16.

FIG. 20 illustrates the engagement member 18 in the second position, with the tissue removal tool 30 removing the tissue sample TS that has been severed by the cutting mechanism 12. In this embodiment, the hook or hook-like end of the tissue sample retainer 32 latches to the tissue sample TS while the tissue T is severed and released by the engagement member 18. Thus, when the tissue sample TS is completely cut from the tissue T, the tissue removal tool 30 is already in position to remove the tissue sample TS and can withdraw the tissue sample TS from the cutting tool 10 by moving axially in the direction D2 away from the cutting mechanism 12. In another embodiment, the tissue removal tool 30 can move in the direction D1 to attach to the tissue sample TS after the tissue sample TS is severed from the tissue T by the cutting mechanism 12.

After the tissue sample TS is removed, the cutting tool 10 can remain attached to the tissue T and serve as a dam, preventing bleeding and air leakage from the tissue T. A suction catheter can be positioned in the pleural space to determine whether bleeding or air leakage from the tissue T is occurring. At least one follow-up CT with the cutting tool 10 and/or with a pleural drainage tube in place can be used to determine whether the suction catheter can be removed. Subcutaneous tissues do not require re-approximation, and access sites can be approximated with steri-strip. The tissue sampling process can be repeated in another region of the same lobe, or in another lobe if necessary.

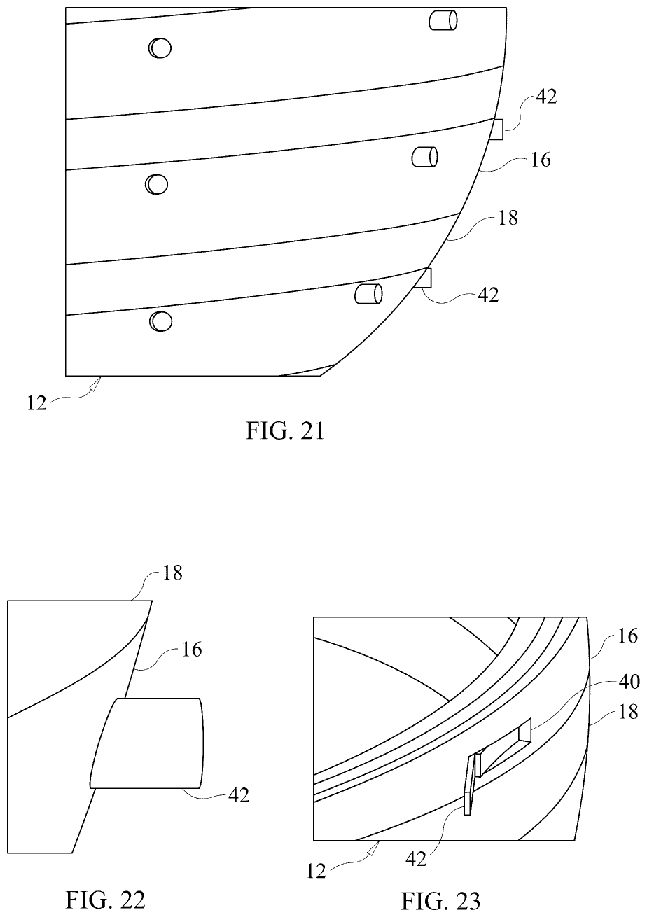

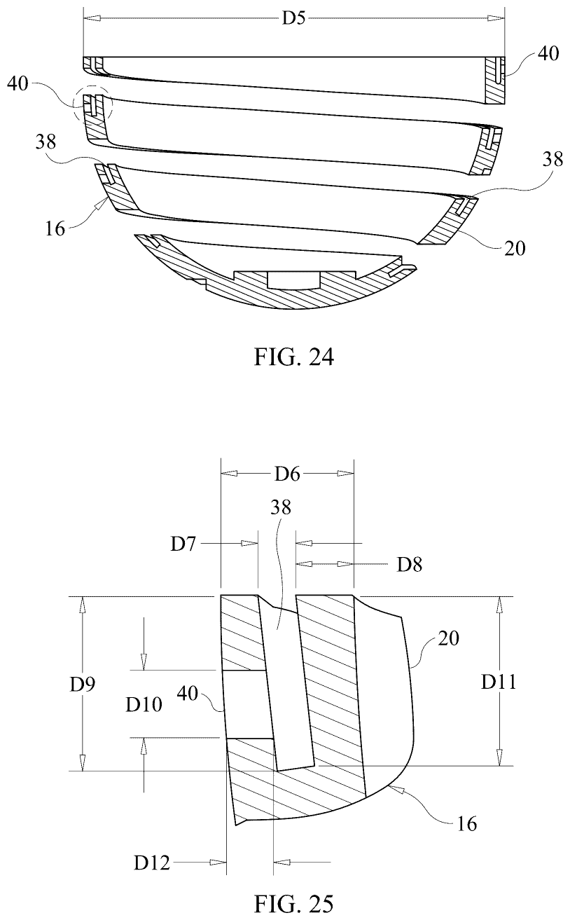

FIGS. 21 to 25 illustrate the cutting member 16 having a plurality of embedded projections 42. As shown in FIGS. 24 and 25, a groove 38 is formed along an upper surface of the strand 20 of the cutting member 16. The groove 38 has a substantially U-shaped cross section. As further shown in FIGS. 23 to 25, a plurality of windows 40 are formed in an outer side surface of the strand 20 of the cutting member 16. An embedded projection 42 projects outwardly through each of the windows 40. The projections 42 can be any suitable size and shape, such as having a barb shape and a length of approximately 1 to 3 mm. The projections 42 can also point in various directions. When the engagement member 18 moves through the groove 38, the engagement member 18 pushes the embedded projections 42 out through the windows 40. The projections 42 penetrate the tissue T, and securely retain the cutting mechanism 12 in the tissue T to substantially prevent the cutting mechanism 12 from being dislodged.

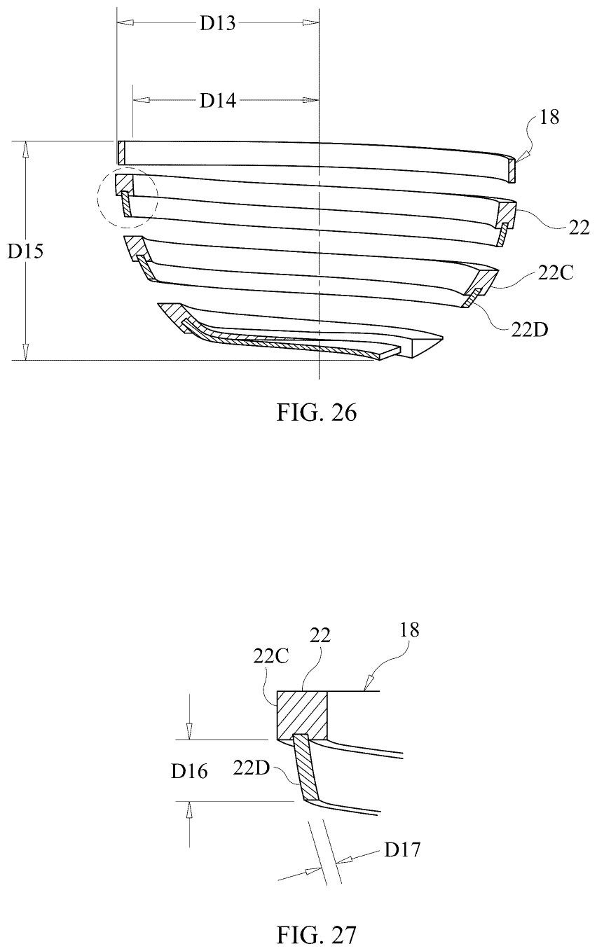

As shown in FIGS. 26 and 27, the strand 22 of the engagement member 18 has a first portion 22C and a second portion 22D. The width of the first portion 22C is larger than the width of the second portion 22D. The first portion 22C fills in the gaps between the successive coils of the cutting member 16. The second portion 22D travels through the groove 38 in the cutting member 16 as the engagement member 18 is moved from the first position to the second position in full engagement with the cutting member 16. The second portion 22D moves through the groove 38 and contacts the embedded projections 42, pushing the projections 42 out through the windows 40, thereby securely retaining the cutting tool 10 in the tissue T as the engagement member 18 engages the cutting member 16. FIG. 26 shows the shape of the engagement member 18 in the second position, after engaging and being conformed to the cutting member 16 to form the completed cutting mechanism 12.

In an embodiment, biocompatible glue-like materials can also be embedded in the cutting member 16 and extruded as the engagement member 18 travels along the cutting member 16 to form the cutting mechanism 12, or within a cavity of the cutting assembly 12, to improve adherence to lung tissue, hemostasis, and pneumostasis. The biopsy device 5 can also include a heating element for cauterization purposes.

The cutting member 16 and the engagement member 18 can be any suitable dimensions. FIGS. 24 to 27 illustrate approximate dimensions for an example embodiment. As seen in FIG. 24, the overall diameter D5 at the larger end of the cutting member 16 connected to the shaft 11 can be approximately 6 mm. As seen in FIG. 25, the strand 20 thickness D6 can be approximately 0.25 mm, the groove 38 thickness D7 can be approximately 0.07 mm, the thickness D8 from the groove to the inner surface of the strand 20 can be approximately 0.11 mm, the groove 38 length D9 on one side can be approximately 0.34 mm, the window 40 length D10 can be approximately 0.13 mm, the groove length D11 on the other side can be approximately 0.33 mm, and the window width D12 can be approximately 0.09 mm. As seen in FIG. 26, the engagement member 18 outer radius D13 can be approximately 3 mm, the engagement member 18 inner radius D14 can be approximately 2.75 mm, and the total engagement member length D15 can be approximately 3.24 mm. As seen in FIG. 27, the second portion 22D length D16 can be approximately 0.33 mm, and the second portion 22D width D17 can be approximately 0.07 mm.

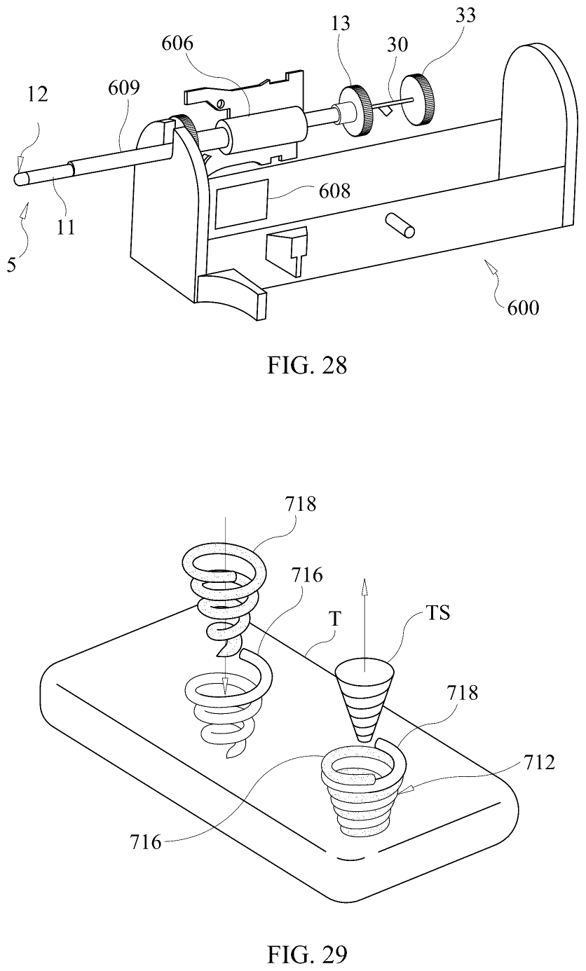

FIG. 28 illustrates an example embodiment of a biopsy system 600 in accordance with the present disclosure. The biopsy system 600 includes the biopsy device 5 as discussed above, along with one or more motor 606, such as a piezo sensor motor, that controls operation of the engagement member 18 to form the cutting mechanism 12. The biopsy system 600 can therefore be used to automatically cause movement of the elements discussed herein to retrieve the tissue sample TS. For example, in the illustrated embodiment, the biopsy system 600 includes a shaft 609 configured to receive and rotate the hollow shaft 11. The biopsy system 600 can thus rotate the cutting member 16 to initially cut into the tissue T, and can then translate and/or rotate the engagement member 18 as discussed above to further sever the tissue sample TS for removal by the sample removal tool 30. The biopsy system 600 can also include additional elements to automatically rotate the handling portion 13 and/or the handling portion 33, or to translate the sample removal tool 30 back and forth within the hollow shaft 11 to withdraw the tissue sample TS as discussed above. In an embodiment, the biopsy system 600 can thus rotate and/or translate one or more of the hollow shaft 11, the handling portion 13, the cutting member 16, the engagement member 18 and the sample removal tool 30 with respect to each other.

In an embodiment, the biopsy system 600 includes a controller 608 configured to control the movement of the cutting tool 10, the cutting member 16, the engagement member 18, the sample removal tool 30 and/or other elements of the biopsy system 600 via the one or more motor 606. The controller 608 preferably includes a microcomputer with a control program that controls one or more of the cutting tool 10, the cutting member 16, the engagement member 18, the sample removal tool 30 and/or other elements. The controller 608 can also include other conventional components such as an input interface circuit, an output interface circuit, and storage devices such as a ROM (Read Only Memory) device and a RAM (Random Access Memory) device. The memory circuit stores processing results and control programs for operation that are run by the processor circuit. The microcomputer of the controller 608 is programmed to control the speed and/or relative movement of the cutting tool 10, the cutting member 16, the engagement member 18, the sample removal tool 30 and/or other elements. The controller 608 is capable of selectively controlling any of the other components of the biopsy system 600 in accordance with one or more control programs. The controller 608 is configured to adjust the speed and/or relative movement, for example, depending on the type of tissue being sampled, the size of the sample, the location and/or other factors.

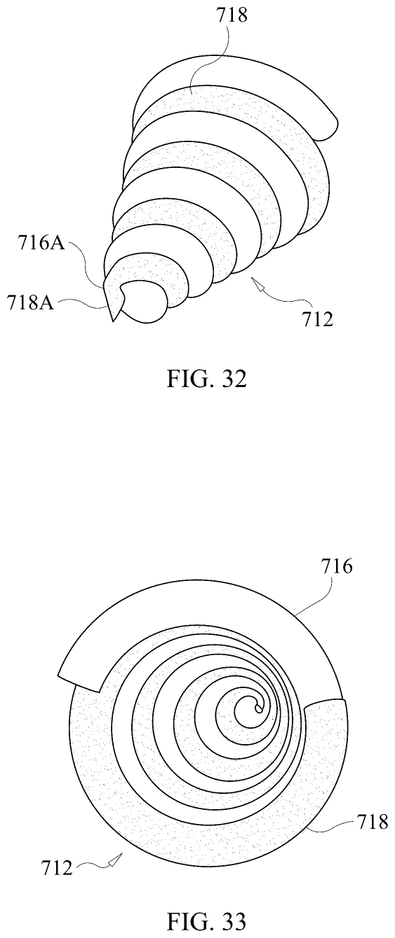

FIGS. 29 to 33 illustrate another example embodiment of a cutting member 716 and an engagement member 718. In this embodiment, the cutting member 716 and the engagement member 718 have substantially identical shapes, shown as conical coils. Distal ends 716A and 718A of each of the cutting member 716 and the engagement member 718 are sharp to facilitate cutting through tissue TS. The cutting member 716 and the engagement member 718 can be made of any suitable material, such as titanium. The cutting member 716 and the engagement member 718 can be any suitable size, such as 1 mm diameter titanium wire.

As shown on the left side of FIG. 29, the cutting member 716 initially cuts into the tissue T. The engagement member 718 then engages the cutting member 716, further cutting the tissue T to sever the tissue sample TS as shown on the right side of FIG. 29. The tissue T is cut along the intersecting wire tangents as the engagement member 718 travels along the cutting member 716 and the cutting member 716 and the second cutting member 716 tighten against one another.

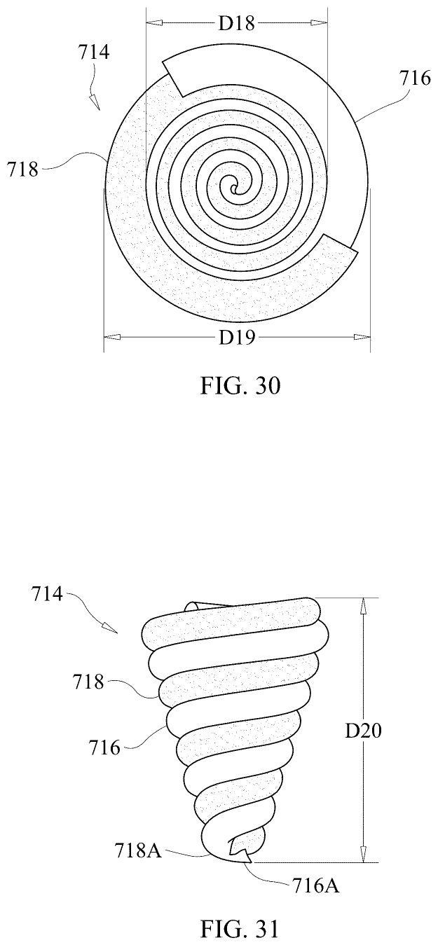

The cutting member 716 and the engagement member 718 can be any suitable dimensions to achieve the desired size of the tissue sample TS. For example, as seen in FIG. 30, the inner diameter D18 of the opening at the proximal end of the cutting mechanism formed by the cutting member 716 and the engagement member 718 is approximately 5 mm, and the outer diameter D19 of the cutting mechanism formed by the cutting member 716 and the second cutting member 716 is approximately 7 mm. As seen in FIG. 31, the length D20 of the cutting mechanism formed by the cutting member 716 and the second cutting member 716 is approximately 10 mm.

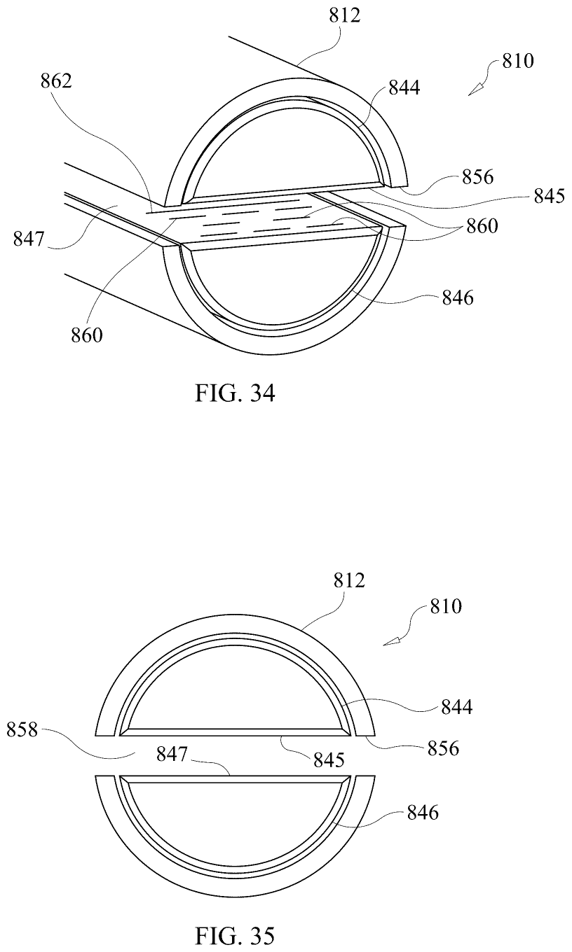

FIGS. 34 to 39 illustrate another example embodiment of a biopsy device 810 configured to sample tissue T in accordance with the present disclosure. In the illustrated embodiment, the biopsy device 810 includes a shaft or port 812. The biopsy device 810 further includes a first gripping member 844 and a second gripping member 846. The first and second gripping members 844 and 846 are movably disposed within the shaft or port 812. The first gripping member 844 includes a first surface 845 facing the second gripping member 846, and the second gripping member 846 likewise has a second surface 847 facing the first gripping member 844. The biopsy device 810 further includes a longitudinally extending recess 856 in the shaft or port 812 which is aligned with a gap 858 between the first surface 845 and the second surface 847.

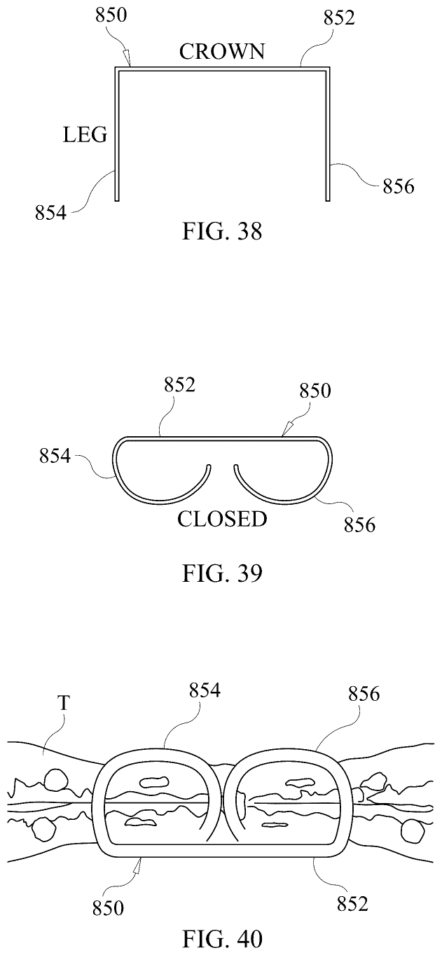

The second gripping member 846 includes a plurality of first openings 860. More specifically, the second surface 847 includes the plurality of first openings 860. Each of the first openings 860 is configured to eject a staple 850, for example, as shown in FIGS. 38 to 40. The second gripping member 846 moves toward the first gripping member 844, or vice versa, to eject the staple. The first gripping member 844 provides resistance to close the staple 850 as seen in FIG. 40.

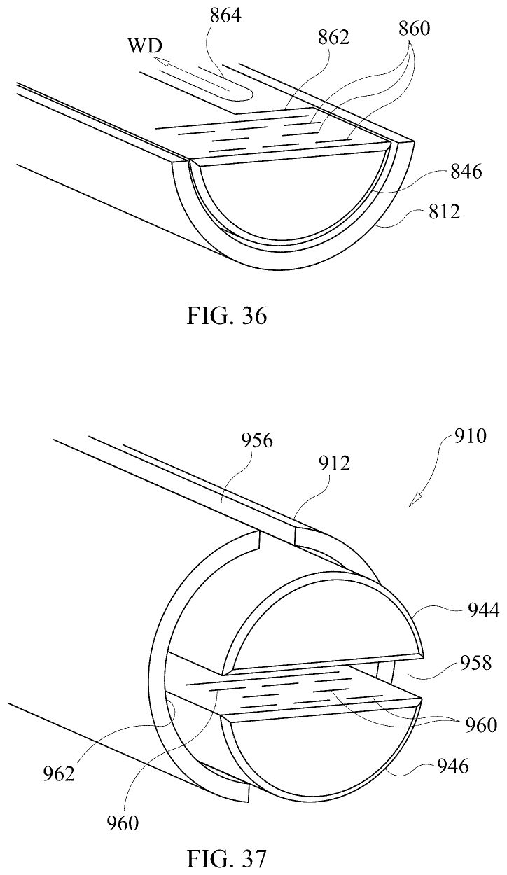

As seen in FIG. 36, the second gripping member 846 includes a second opening 862 rearward of the plurality of first openings 860. A cutting member 864, such as a blade, is pushed out through the second opening 862 when one or both of the first gripping member 844 and the second gripping member 846 is moved towards the other. The cutting member 864 is configured to sever a tissue sample TS from the tissue T held by the first and second gripping members 844 and 846.

Movement of the first gripping member 844 and/or the second gripping member 846 both severs the tissue sample TS with the cutting member 864 pushed out through the second opening 862 and ejects the staple(s) 850 through the first opening(s) 860 into the tissue T. In other words, one motion of moving the first gripping member 844 and/or the second gripping member 846 toward each other both severs the tissue sample TS with the cutting member 864 and ejects the staple 850 to close the site.

The staples 850 are disposed in the biopsy device 810. As shown in FIGS. 38 to 40, a staple 850 has a crown 852 and legs 854, 856 extending from opposite ends of the crown 852 to form a substantially U-shaped staple 850. When the staple 850 is ejected from one or both of the first and second gripping members 844, 846, the staple 850 contacts the other of the first and second gripping members 844, 846 to collapse the first and second legs 854, 856 toward the crown 852. The inserted staple 850 substantially prevents spring back of the tissue 824 and maintains the tissue 824 in a compressed state. In the illustrated embodiment, the staple 850 has a height of approximately 1.5 mm, although the staple 850 can have any suitable height depending on the application.

The biopsy device 810 facilitates efficiently obtaining a tissue sample TS and closing the site of the tissue sample TS with a single device. The biopsy device 810 obtains a tissue sample TS, and then closes the site of the tissue sample TS with a staple 850. As seen in FIG. 40, the inserted staple 850 prevents blood and air from escaping from the site of the tissue sample, such as a lung.

In an embodiment, a tissue removal tool 30, 130, 230, 330, 430, 530 as described above, or a similar tissue removal tool, can be located within the shaft or port 812 as the tissue sample TS is cut by the cutting member 864. As seen in FIG. 36, the tissue removal tool 30, 130, 230, 330, 430, 530 can be configured to withdraw the tissue sample TS in the withdrawal direction WD after the movement of the first gripping member 844 and the second gripping member 846 causes the cutting member 864 to sever the tissue sample TS from the tissue T. The tissue sample TS can be secured by a tissue sample retainer 32, 132, 232, 332, 432, 532 as described above and withdrawn as described above from the opposite end of the hollow shaft or port 812 from the first gripping member 844 and the second gripping member 846.

FIG. 37 illustrates an alternative exemplary embodiment of a biopsy device 910 configured to sample tissue T. The biopsy device 910 is substantially similar to the biopsy device 810 illustrated in FIGS. 34 to 36. In the illustrated embodiment, the biopsy device 910 includes a first gripping member 944 and a second gripping member 946. The first and second gripping members 944, 946 are movably disposed within a shaft or port 912. A longitudinally extending recess 956 in the shaft or port 912 is offset from a gap 958 between the first and second gripping members 944, 946. The longitudinally extending recess 956 is offset by 90 degrees from the gap 958 between the first and second gripping members 944, 946. Movement of the first gripping member 946 toward the second gripping member 948 ejects the staples through the first openings 960 into the tissue T and severs the tissue sample TS with the cutting member pushed out through the second opening 962. In other words, like the previous embodiment, one motion of moving one or both of the first gripping member 946 and the second gripping member 948 towards the other both ejects the staples 850 to close the site and severs the tissue sample TS with the cutting member.

A biopsy device in accordance with the present disclosure reduces invasiveness associated with tissue sampling. Samples of lung tissue ranging from 5 mm to 7 mm to 1 cm in diameter can be obtained through small incisions, with CT guidance, by the interventional radiology (IR) team, using local analgesia only, or conscious sedation if necessary. Post procedure pain control likely will not require narcotic medication.

A biopsy device in accordance with the present disclosure improves safety of tissue sampling. Safety is improved by decreased invasiveness overall, specifically by using smaller incisions, avoiding general anesthesia, avoiding double lumen endotracheal intubation, and reducing the length of hospitalization. Lung diseases are usually discovered, and diagnosed, at later stages of the illness, and the systems and methods disclosed herein provide a less invasive diagnostic procedure which helps to facilitate definitive diagnoses in patients who would have otherwise been considered too high risk for an open lung biopsy procedure. Physicians can refer more patients earlier for a less invasive diagnostic procedure, thereby avoiding delayed or empiric treatment.

A biopsy device in accordance with the present disclosure provides a less expensive tissue sampling procedure. The procedure obviates the need for a longer and more dangerous operation in the operating room, reduces the need for hospitalization and the length of hospitalization, reduces the need for costly equipment and personnel, and reduces costly complications and readmissions. Additionally, by diagnosing and potentially treating a large population of citizens earlier, the societal cost of citizens out of work and patients suffering from chronic illness are reduced, and quality of life is improved.

### General Interpretation of Terms

The foregoing detailed description of the certain exemplary embodiments has been provided for the purpose of explaining the principles of the invention and its practical application, thereby enabling others skilled in the art to understand the invention for various exemplary embodiments and with various modifications as are suited to the particular use contemplated. This description is not necessarily intended to be exhaustive or to limit the invention to the exemplary embodiments disclosed. Any of the exemplary embodiments and/or elements disclosed herein may be combined with one another to form various additional embodiments not specifically disclosed. Accordingly, additional embodiments are possible and are intended to be encompassed within this specification and the scope of the appended claims. The specification describes specific examples to accomplish a more general goal that may be accomplished in another way.

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts unless otherwise stated.

Also, it will be understood that although the terms “first” and “second” may be used herein to describe various components, these components should not be limited by these terms. These terms are only used to distinguish one component from another. Thus, for example, a first component discussed above could be termed a second component and vice versa without departing from the teachings of the present invention. The term “attached” or “attaching”, as used herein, encompasses configurations in which an element is directly secured to another element by affixing the element directly to the other element; configurations in which the element is indirectly secured to the other element by affixing the element to the intermediate member(s) which in turn are affixed to the other element; and configurations in which one element is integral with another element, i.e. one element is essentially part of the other element. This definition also applies to words of similar meaning, for example, “joined”, “connected”, “coupled”, “mounted”, “bonded”, “fixed” and their derivatives. Finally, terms of degree such as “substantially”, “about” and “approximately” as used herein mean an amount of deviation of the modified term such that the end result is not significantly changed.

While only selected embodiments have been chosen to illustrate the present invention, it will be apparent to those skilled in the art from this disclosure that various changes and modifications can be made herein without departing from the scope of the invention as defined in the appended claims. For example, unless specifically stated otherwise, the size, shape, location or orientation of the various components can be changed as needed and/or desired so long as the changes do not substantially affect their intended function. Unless specifically stated otherwise, components that are shown directly connected or contacting each other can have intermediate structures disposed between them so long as the changes do not substantially affect their intended function. The functions of one element can be performed by two, and vice versa unless specifically stated otherwise. The structures and functions of one embodiment can be adopted in another embodiment. It is not necessary for all advantages to be present in a particular embodiment at the same time. Every feature which is unique from the prior art, alone or in combination with other features, also should be considered a separate description of further inventions by the applicant, including the structural and/or functional concepts embodied by such feature(s). Thus, the foregoing descriptions of the exemplary embodiments according to the present invention are provided for illustration only, and not for the purpose of limiting the invention as defined by the appended claims and their equivalents.

What is claimed is:

1. A biopsy device comprising:
a hollow shaft;
a cutting member connected to an end of the hollow shaft and configured to cut into tissue; and
an engagement member movably connected to the hollow shaft and being configured to move rotationally and axially relative to the cutting member, the engagement member configured to move from a first position to a second position engaging the cutting member after the cutting member has initially engaged the tissue.

2. The biopsy device of claim 1, wherein
the cutting member has a substantially helical shape with at least one axial gap being defined between adjacent coils.

3. The biopsy device of claim 2, wherein
the engagement member has a first shape in the first position and a second shape in the second position, the second shape being different from the first shape.

4. The biopsy device of claim 2, wherein
the engagement member rotates into the at least one axial gap when engaging the cutting member in the second position.

5. The biopsy device of claim 1, wherein
the cutting member includes a strand having at least one groove in a surface thereof, and
a portion of the engagement member travels through the groove as the engagement member engages the cutting member.

6. The biopsy device of claim 1, further comprising
a sample removal tool configured to translate axially within the hollow shaft to withdraw a tissue sample severed from tissue by at least the cutting member.

7. The biopsy device of claim 1, wherein
the cutting member includes one or more anchor protrusion that protrudes outwardly from the cutting member when the engagement member is disposed in the second position

8. The biopsy device of claim 7, wherein
the engagement member pushes the one or more anchor protrusion outwardly from the cutting member when the engagement member engages the cutting member.

9. The biopsy device of claim 1, comprising
a motor configured to control movement of the engagement member with respect to the cutting member.

10. A method of performing a biopsy, the method comprising
cutting into tissue with a cutting member attached to a hollow shaft at a location of a desired tissue sample;
moving an engagement member into the engagement with the cutting member so as to further sever the tissue sample from the tissue; and
withdrawing the tissue sample through the hollow shaft with a tissue removal tool after the tissue sample is severed from the tissue by interaction of the cutting member and the engagement member.

11. The method of claim 10, comprising
cutting into the tissue with the cutting member encircling a tissue sample retainer of the tissue removal tool.

12. The method of claim 10, wherein
moving the engagement member into engagement with the cutting member includes rotating the engagement member into the cutting member.

13. The method of claim 10, wherein
moving the engagement member into engagement with the cutting member includes moving the engagement member into a gap between adjacent coils of the cutting member.

14. The method of claim 10, comprising
anchoring the cutting member in the tissue before withdrawing the tissue sample.

15. A biopsy device comprising:
a first gripping member;
a second gripping member separated from the first gripping member by an axially extending gap;
a cutting tool adjacent one of the first gripping member and the second gripping member; and
at least one staple being disposed in one of the first and second gripping members,
the first gripping member and the second gripping member positioned and arranged such that movement of at least one of the first gripping member and the second gripping member toward the other with tissue located in the axially extending gap both removes a tissue sample from the tissue and ejects the at least one staple into the tissue.

16. The biopsy device of claim 15, wherein
the first gripping member includes a first surface,
the second gripping member includes a second surface facing the first surface, and
the axially extending gap is located between the first surface and the second surface.

17. The biopsy device of claim 16, wherein
the at least one staple is ejected from one of the first surface or the second surface, and
the other of the first surface and the second surface collapses the at least one staple into the tissue.

18. The biopsy device of claim 15, comprising
a hollow shaft containing the first gripping member and the second gripping member.

19. The biopsy device of claim 18, comprising
a tissue removal tool configured to withdraw the tissue sample from the hollow shaft after the movement of the first gripping member and the second gripping member severs the tissue sample from the tissue.

20. The biopsy device of claim 19, wherein
the first gripping member and the second gripping member are located at one end of the hollow shaft, and the tissue removal tool is configured to withdraw the tissue sample from an opposite end of the hollow shaft.
